(19) United States
(12) Patent Application Publication
(10) Pub. No.: US 2026/0124145 A1
(43) Pub. Date: May 7, 2026

(54) LITHIUM SALT EXTENDED-RELEASE FORMULATIONS; METHODS OF MAKING; AND METHODS OF USE THEREOF

(71) Applicant: Almatica Pharma LLC, Morristown, NJ (US)

(72) Inventors: Ripal Gaudana, Morristown, NJ (US); Raghav Gupta, Morristown, NJ (US); Rama Yarasani, Morristown, NJ (US)

(21) Appl. No.: 19/442,284

(22) Filed: Jan. 7, 2026

Related U.S. Application Data
(63) Continuation-in-part of application No. 18/751,410, filed on Jun. 24, 2024.
(60) Provisional application No. 63/522,883, filed on Jun. 23, 2023.

(51) Int. Cl. A61K 9/20; A61K 9/00; A61K 33/00

(52) U.S. Cl. CPC A61K 9/2086; A61K 9/0065; A61K 9/2027; A61K 9/2031; A61K 9/2054; A61K 9/2077; A61K 33/00

(57) ABSTRACT

Disclosed are high dose gastric retentive, extended-release lithium salt dosage forms suitable for once a day administration.

### CROSS-REFERENCE TO RELATED APPLICATIONS

This application is a continuation-in-part of U.S. patent application Ser. No. 18/751,410, filed Jun. 24, 2024, which claims the benefit of U.S. Provisional Application No. 63/522,883, filed Jun. 23, 2023, each of which is hereby incorporated by reference in its entirety for all purposes.

### BACKGROUND

Lithium carbonate was originally approved in the United States (U.S.) in 1970 as a 300 mg oral capsule. Other solid oral dosage forms of immediate-release lithium carbonate (i.e., tablets) as well as an oral syrup (as lithium citrate) have also been approved and marketed in the U.S. Extended-release lithium carbonate products have been approved in the U.S., including ESKALITH CR® (450 mg lithium carbonate extended-release tablet; NDA 018152; discontinued), LITHOBID® (300 mg lithium carbonate extended-release tablet; NDA 018027). LITHOBID and the ANDA-approved 450 mg lithium carbonate extended-release tablet product marketed by Hikma Pharmaceuticals USA Inc. (ANDA 076691).

Lithium is recognized as having a narrow therapeutic index (NTI) (Drug Bank DBCAT003972), and the prescribing information for lithium carbonate products marketed in the U.S. carry a Black Box Warning regarding lithium toxicity being closely related to serum lithium levels and which can occur at doses close to therapeutic levels. The therapeutic range for lithium is identified as 0.8-1.2 mEq/L and the toxic concentration of lithium is understood to be >1.5 mEq/L.

Currently for treatment of acute mania 1800 mg of lithium carbonate per day is prescribed and the same is achieved by administering 3 tablets of LITHOBID® ER 300 mg in the morning and 3 tablets in the evening. Thus, patients need to take 6 tablets, while for long term control, 900-1200 mg/day is required. Again, this requires administration of multiple tablets as well as twice daily dosing using currently approved products. Using the 450 mg extended-release tablet also requires multiple tablets and twice daily dosing to achieve 900 mg and greater strengths required for either long term control or acute mania. Due to lithium having a NTI, close monitoring of patients serum level lithium is required and the existing immediate release formulations are prescribed for closer titration.

Therefore, there remains a need in the art for a higher strength lithium salt formulation that exhibits an extended-release profile for reduced number of administrations per day with fewer dosage units per administration for simpler and more convenient dosing.

### SUMMARY

Disclosed herein is an extended-release oral tablet, comprising an extended-release polymer matrix portion comprising lithium carbonate and a controlled-release polymer; and a gastroretentive portion comprising a swellable polymer; wherein the tablet contains greater than 450 mg lithium carbonate per tablet. In an embodiment, the tablet is in the form of a layered tablet comprising a first layer comprising the extended-release polymer matrix portion and a second layer comprising the gastroretentive portion. In an embodiment, the tablet comprises 900 mg lithium carbonate.

In another embodiment, a method of preparing an extended-release tablet comprises providing a first blend comprising lithium carbonate and a controlled-release polymer; providing a second blend comprising a swellable polymer; and compressing the first blend and the second blend to form a bilayer tablet comprising greater than 450 mg lithium carbonate per tablet. In an embodiment, the tablet comprises 900 mg lithium carbonate.

In another embodiment, a method of treating a subject in need thereof comprises administering the tablet described herein to a subject in need thereof to treat depression or mania.

The above described and other features are exemplified by the following detailed description.

### DETAILED DESCRIPTION

Disclosed herein are higher strength, extended-release oral lithium salt formulations that require fewer doses per day than immediate release or currently available extended release tablet formulations, which require multiple doses per day. The formulation satisfies an unmet medical need for a once a day oral lithium dosage form and would greatly benefit patients currently taking multiple tablets twice a day. Patient compliance is improved with a reduced number of administrations and reduced pill burden.

In an embodiment, the lithium salt is lithium carbonate and the formulation is a gastroretentive, extended release tablet formulation providing therapeutic lithium levels for 24 hours. It has been found that a layered tablet comprising a first layer comprising the extended-release polymer matrix portion and a second layer comprising the gastroretentive portion advantageously provides 24 hour extended release due to the absorption window of lithium. A single layer extended release/gastroretentive formulation did not work well as it slows down the dissolution. A single layer extended release/non gastroretentive formulation does not work well as it exits the absorption window for lithium prematurely. The layered tablet comprising a first layer comprising the extended-release polymer matrix portion and a second layer comprising the gastroretentive portion provides the necessary retention and release profile required for a 24 hour dosing.

An extended-release oral tablet comprises an extended-release polymer matrix portion comprising lithium carbonate and a controlled-release polymer; and a gastroretentive portion comprising a swellable polymer, wherein the tablet contains greater than 450 mg lithium carbonate per tablet. Specifically, the tablet contains about 500 mg or more of lithium carbonate per tablet, about 600 mg or more of lithium carbonate, about 700 mg or more of lithium carbonate, about 800 mg or more of lithium carbonate, or about 900 mg lithium carbonate per tablet.

The extended-release polymer matrix portion comprising lithium carbonate and a controlled-release polymer provides extended release of lithium through erosion and diffusion. The gastroretentive portion comprises a swellable polymer that swells and promotes gastroretention of the dosage form in the stomach. This allows the dosage form to remain longer within its optimum absorption window in the gastrointestinal tract, thereby providing better absorption of the active. The use of the gastroretentive portion allows for the dosage form to remain in the stomach for a longer duration of time than a corresponding dosage form without the gastroretentive portion. The extended time in the stomach allows for the lithium to be fully released and to get absorbed completely.

The gastroretentive portion can be formulated to tailor the degree of retention in the stomach by adjusting size of the portion, type and amount of swellable polymer (average weight, size), use of different excipients, etc. The gastroretentive portion and swellable polymers are selected to provide rapid swelling while exhibiting enough gel strength to withstand the fed stomach conditions.

By “once-a-day administration” is meant administration once within a 24 hour period.

In certain embodiments, the extended-release polymer matrix portion of the extended-release oral tablet comprises lithium carbonate and a controlled-release polymer, wherein the lithium carbonate can be present in an amount of about 60 to about 90 weight percent (wt %) based on the total weight of the extended-release polymer matrix portion, specifically about 65 to about 85 wt %, and yet more specifically about 70 to about 80 wt %.

Suitable controlled-release polymers for use in the extended-release polymer matrix portion include hydroxypropyl methyl cellulose, including high molecular weight hydroxypropyl methyl cellulose having a molecular weight of about 4,000 to about 1,500,000, or 25,000 and above. Suitable commercially available controlled-release grades of hydroxypropyl methyl cellulose include those available from Colorcon and International Flavors and Fragrances Inc., specifically the METHOCEL Premium CR Grades (see Table 1). A single controlled-release grade of hydroxypropyl methyl cellulose can be used, or a combination of hydroxypropyl methyl cellulose of varying grades can be used (e.g., combination of METHOCEL K4M CR and K100 LV, or K15M CR and K100 LV).

TABLE 1

| | METHOCEL Premium Product Grade | | | | | |
|---|---|---|---|---|---|---|
| | K100 | K4M | K15M | K100M | E4M | E10M |
| | Premium | Premium | Premium | Premium | Premium | Premium |
| | LV CR | CR | CR | CR | CR | CR |
| Methoxyl, % (USP) | 19-24 | 19-24 | 19-24 | 19-24 | 28-30 | 28-30 |
| Hydroxypropoxyl, % | 7-12 | 7-12 | 7-12 | 7-12 | 7-12 | 7-12 |
| (USP) | | | | | | |
| Substitution type | 2208 | 2208 | 2208 | 2208 | 2910 | 2910 |
| (USP/EP) | | | | | | |
| Apparent viscosity, | 80-120 | 3000-5600 | 11250-21000 | 80000-120000 | 3000-5600 | 7500-14000 |
| 2% in water at | | | | | | |
| 20° C., cP (USP) | | | | | | |
| Apparent viscosity, | 78-117 | 2308-3755 | 6138-9030 | 16922-19267 | 2308-3755 | 4646-7070 |
| 2% in water at | [98 Nom] | [2903 Nom] | [7382 Nom] | [18243 Nom] | [2903 Nom] | [5673 Nom] |
| 20° C., mPa · s (EP) | | | | | | |

The controlled-release polymer for use in the extended-release polymer matrix portion may also be a high molecular weight polyethylene oxide having an approximate molecular weight of 100,000 or greater based on dilute viscosity measurements, specifically about 200,000, about 300,000, about 400,000, about 600,000, about 900,000, about 1,000,000, about 2,000,000, about 4,000,000, about 5,000,000, about 7,000,000, etc. Exemplary swellable polymers for use in the extended release portion include the POLYOX WSR polymers and the polymers in Table 2.

TABLE 2

| | | Viscosity of aqueous | |
|---|---|---|---|
| | Approximate | solution at 25° C. | |
| | Molecular | and given concentration, | |
| Grade | Weight¹ | Wt % | mPa · s (cP) |
| POLYOX ™ WSR N-10 | 100,000 | 5 | 12-50 |
| POLYOX ™ WSR N-80 | 200,000 | 5 | 65-115 |
| POLYOX ™ WSR N-750 | 300,000 | 5 | 600-1,000 |
| POLYOX ™ WSR N-3000 | 400,000 | 5 | 2,250-4,500 |
| POLYOX ™ WSR 205 | 600,000 | 5 | 4,500-8,800 |
| POLYOX ™ WSR 1105 | 900,000 | 5 | 8,800-17,600 |
| POLYOX ™ WSR N-12K | 1,000,000 | 2 | 400-800 |
| POLYOX ™ WSR N-60K | 2,000,000 | 2 | 2,000-4,000 |
| POLYOX ™ WSR 301 | 4,000,000 | 1 | 1,650-5,500 |
| POLYOX ™ WSR | 5,000,000 | 1 | 5,500-7,500 |
| Coagulant | | | |
| POLYOX ™ WSR 303 | 7,000,000 | 1 | 7,500-10,000 |
| POLYOX ™ WSR 308 | 8,000,000 | 1 | 10,000-15,000 |
| UCARFLOC ™ Polymer | 7,500,000 | 1 | 7,500-13,000 |
| 304 CP² | | | |
| UCARFLOC ™ Polymer | 8,500,000 | 1 | >13,000 |
| 309 CP² | | | |

¹Based on rheological measurements. Molecular weights obtained by other methods, including light scattering and gel permeation chromatography, may not be directly comparable.
²Coarse Particle (CP) grade specified as less than 15% through a 200 mesh screen (74 μm).

The controlled-release polymer or combination of controlled-release polymers can be present in the extended-release polymer matrix portion in an amount of about 8 to about 40 wt % based on the total weight of the extended-release polymer matrix portion, specifically about 10 to about 30 wt %, and yet more specifically about 12 to about 20 wt %.

The tablet comprises a gastroretentive portion responsible for extensive swelling to promote retention of the dosage form in the stomach allowing the lithium to be released fully and to get absorbed completely. The gastroretentive portion is formulated to result in fast swelling while exhibiting enough gel strength to withstand the fed stomach conditions and allow sufficient gastric retention for complete absorption of the lithium salt.

The swellable polymer for use in the gastroretentive portion of the dosage form may be any suitable water-swellable or water-soluble polymer including a high molecular weight polyethylene oxide; a cellulose ether such as a hydroxypropyl methyl cellulose, hydroxypropyl cellulose, hydroxyethyl cellulose, and the like; a cellulose such as ethyl cellulose, methyl cellulose, sodium carboxy methyl cellulose; a carbomer (acrylic acid polymer); acacia; pectin; agar; gellan gum; guar gum; an alginate or alginic acid; acrylic acid derivatives such as polycarbophil (acrylic acid polymer crosslinked with divinyl glycol), and the like; chitosan; a combination thereof, and the like.

The swellable polymer may be a high molecular weight polyethylene oxide having an approximate molecular weight of 100,000 or greater based on dilute viscosity measurements, specifically about 200,000, about 300,000, about 400,000, about 600,000, about 900,000, about 1,000,000, about 2,000,000, about 4,000,000, about 5,000,000, about 7,000,000, etc. Exemplary swellable polymers for use in the gastroretentive portion include the POLYOX WSR polymers.

The swellable polymer or combination of swellable polymers make up the bulk of the gastroretentive portion. The swellable polymer can be present in the gastroretentive portion in an amount of greater than about 50 wt % and up to 100 wt % by weight of the gastroretentive portion, specifically about 75 to about 99.5 wt %, and more specifically about 85 to about 99 wt %.

The gastroretentive portion can further comprise a gas generating agent. Exemplary gas generating agents include a carbonate source including carbonate and bicarbonate salts. Suitable carbonate sources include an alkali or alkaline earth metal carbonate, an alkali or alkaline earth metal hydrogen carbonate, a combination thereof, and the like. In an embodiment, the gas generating agent is sodium hydrogen carbonate.

The gastroretentive portion can further comprise an excipient to promote rapid hydration such as a disintegrant (e.g., crospovidone), a channel forming agent (e.g. Eudragit® EPO), a combination thereof, and the like. Exemplary channel forming agents include a cationic copolymer based on dimethylaminoethyl methacrylate, butyl methacrylate, and methyl methacrylate random copolymer. These cationic copolymers are referred to as Amino Methacrylate Copolymer—NF according to USP/NF (United States Pharmacopeia (USP) and the National Formulary (NF)), Basic Butylated Methacrylate Copolymer of European Pharmacopoeia (Ph. Eur.), and Aminoalkyl Methacrylate Copolymer E of Japanese Pharmacopoeia (JPE). Commercially available cationic copolymer based on dimethylaminoethyl methacrylate, butyl methacrylate, and methyl methacrylate includes Evonik Industries' Eudragit® EPO, Eudragit® E 100, Eudragit® E 12,5, or a combination thereof having a ratio of dimethylaminoethyl methacrylate, butyl methacrylate, and methyl methacrylate of 2:1:1.

The tablets may further comprise one or more suitable tablet excipients, such as a binder, a filler, a lubricant, a glidant, a colorant, a disintegrant, or any combination thereof. The one or more suitable tablet excipients may be present in the extended-release polymer matrix portion, the gastroretentive portion, or both. In certain embodiments, the tablet excipient can be an intragranular ingredient with the active agent, an extragranular ingredient blended with active agent granules, or a combination thereof.

Suitable binders for use in the tablets include polyvinylpyrrolidone; a cellulosic polymer including hydroxypropyl cellulose, hydroxyethyl cellulose, methylcellulose, ethyl cellulose, and the like; gum Arabic; alginic acid and its derivatives; a sugar or sugar alcohol such as mannitol, lactose, and the like; a starch; or a combination thereof. In certain embodiments, the binder is an intragranular ingredient. In one aspect, the binder is polyvinylpyrrolidone.

Suitable disintegrants include crospovidone, croscarmellose sodium, sodium starch glycolate, a starch, or a combination thereof.

Suitable fillers include a water insoluble filler, such as dicalcium phosphate, starch, powdered cellulose, microcrystalline cellulose, and the like. Exemplary water-soluble fillers include water soluble sugars and sugar alcohols, specifically lactose, glucose, fructose, sucrose, mannose, dextrose, galactose, mannitol, sorbitol, xylitol, and the like. Combinations of fillers may be used. In certain embodiments, the filler is an intragranular ingredient. In certain embodiments, the filler is an extragranular ingredient.

Exemplary lubricants include talc, calcium stearate, magnesium stearate, solid polyethylene glycols, sodium lauryl sulfate, glyceryl behenate, a combination thereof, and the like. In one aspect, the lubricant is magnesium stearate.

Exemplary glidants include colloidal silicon dioxide, starch, talc, a combination thereof, and the like.

In certain embodiments, the gastroretentive portion is substantially free or free of a lithium salt. As used herein, substantially free of a lithium salt means the gastroretentive portion contains less than 10 wt %, specifically less than 5 wt %, and more specifically less than 1 wt % of a lithium salt based on the total weight of the gastroretentive portion.

The weight ratio of the extended-release polymer matrix portion to the gastroretentive portion can be about 5: 1 to about 1: 1, specifically about 4.5: 1 to about 1.5: 1, more specifically about 4: 1 to about 2: 1, yet more specifically about 3.5: 1 to about 2.5: 1, and still yet more specifically about 3: 1.

The tablet formulations herein may optionally further comprise a non-controlled-release coating, that is, an immediate-release coating, a color-identifying coating, a cosmetic coating, a seal coating, or the like. The non-controlled-release coating, optionally referred to as a non-functional coating, should not have an impact on the release of the active agent due to the initial dissolution, hydration, perforation of the coating, etc., and would not be considered to be a significant deviation from the non-coated formulation. Suitable non-controlled-release coating materials include immediate release film coating systems commercially available by COLORCON under the name OPADRY Non-controlled-release coatings can include a water-soluble polymer, plasticizer, and optionally a pigment or colorant.

In an embodiment, the extended-release tablet is not an effervescent formulation and does not comprise an effervescent or gas generating component.

The extended-release oral tablets can be administered once daily to a subject in need thereof to treat depression or mania. In an embodiment, the extended-release oral tablets can be administered once daily with food or in the fed state to a subject in need thereof to treat depression or mania. When administered with food, the tablet can be administered at the same time as the consumption of food, substantially at the same time as the food, or within about 30 minutes before or after the food, specifically about 20 minutes, more specifically about 15 minutes, yet more specifically about 10 minutes, and still yet more specifically about 5 minutes before or after the food. In further embodiments, the tablet can be administered at the same time as the consumption of food, substantially at the same time as the food, or within about 30 minutes after the food, specifically about 20 minutes, more specifically about 15 minutes, yet more specifically about 10 minutes, and still yet more specifically about 5 minutes after the food “Food” typically means a solid food or mixed solid/liquid food with sufficient bulk and fat content that it is not rapidly dissolved and absorbed in the stomach. In a further embodiment, the food is a high fat meal, high calorie meal, or high fat and high calorie meal.

A “high fat” meal generally means about 30% or more of the total caloric content of the meal is fat, specifically about 40% or more, and yet more specifically about 50% or more. A “high calorie” meal means a meal of about 600 to about 1200 calories, more specifically about 800 to about 1000 calories. In an embodiment, the food is a high-fat and high-calorie meal described as a test meal for food-effect bioavailability and fed bioequivalence studies as set out in the U.S. Food and Drug Administration's Guidance for Industry, Food-Effect Bioavailability and Fed Bioequivalence Studies, U.S. Department of Health and Human Services, Food and Drug Administration, Center for Drug Evaluation and Research (CDER), December 2002. The test meal can derive approximately 150, 250, and 500-600 calories from protein, carbohydrate, and fat, respectively. An example test meal according to the Guidance would be two eggs fried in butter, two strips of bacon, two slices of toast with butter, four ounces of hash brown potatoes and eight ounces of whole milk. Substitutions of this test meal can be made with a meal that provides a similar amount of calories from protein, carbohydrate, and fat and has comparable meal volume and viscosity.

In an embodiment, the administration of the tablets in the fed state results in enhanced bioavailability of the lithium salt compared to in the fasted state. In an embodiment, the administration of the tablets in the fed state results in an increase in Cₘₐₓ, AUC₀₋ₜ, AUC_{0-∞}, or a combination thereof compared to in the fasted state. The area under the plasma concentration-time curve from time zero to the time of measurement of the last quantifiable concentration (AUC₀₋ₜ) and to infinity (AUC_{0-∞}), peak plasma concentration (Cₘₐₓ), and time to peak concentration (Tₘₐₓ) can be determined according to standard techniques.

The features and advantages are more fully shown by the following examples, which are provided for purposes of illustration and are not to be construed as limiting the invention in any way.

### EXAMPLES

### Example 1. Extended-Release Lithium Carbonate Matrix Tablet (Non Gastro-Retentive)

Example 1.

| Component | % |
|---|---|
| Lithium carbonate | 71.4 |
| Povidone | 3.0 |
| Microcrystalline cellulose (AVICEL PH 101) | 12.7 |
| Hypromellose (HPMC K15M Premium CR) | 6.0 |
| Hypromellose (HPMC K100LV CR) | 5.6 |
| Colloidal silicon dioxide (Cab-O-Sil, M5P) | 0.5 |
| Magnesium stearate | 0.8 |

Povidone binder solution was prepared in purified water. Lithium carbonate (API), some part of microcrystalline cellulose and hypromellose (HPMC K15M Premium CR) were mixed in High-Shear Mixer (GMX) for dry mixing and granulated using the povidone binder solution. Then the granulation was dried using fluid bed dryer until the loss on drying (LOD) was less than or equal to 1%. The dried granulation was milled through Fitzmill. The milled granulation was blended with extra-granular materials such as remaining microcrystalline cellulose, hypromellose (K100 LV CR), colloidal silicon dioxide. The blend was lubricated using magnesium stearate using the same blender. Finally, single layer tablets were compressed using rotary tablet press as per target tablet weight. The core tablets were then film-coated using an Opadry II film coating.

### Examples 2-5. Extended-Release Lithium Carbonate Matrix Bilayer Tablet—Gastroretentive Formulation

| | Example 2. | Example 3. | Example 4. | Example 5. |
|---|---|---|---|---|
| | % | | | |
| API Granules | | | | |
| Lithium carbonate | 58.3 | 61.9 | 62.5 | 62.5 |
| Povidone | 2.3 | 2.5 | 2.5 | 2.5 |
| Extended release Blend | | | | |
| Microcrystalline cellulose (AVICEL PH 101) | 3.9 | — | 4.2 | 4.2 |
| Polyethylene oxide (WSR 205 grade) | — | 8.3 | — | — |
| Hypromellose (HPMC K4M Premium CR) | 7.8 | 2.1 | 6.3 | 8.3 |
| Hypromellose (HPMC K 100 LV) | 3.6 | — | — | 0.0 |
| Hypromellose (HPMC K100 Premium LV CR) | — | — | 5.9 | 3.8 |
| Colloidal silicon dioxide | 0.3 | — | 0.3 | 0.3 |
| Magnesium stearate | 0.6 | 0.6 | 0.7 | 0.7 |
| Gastroretentive Layer | 0.0 | 0.0 | 0.0 | 0.0 |
| Polyethylene oxide (WSR 303) | 18.2 | 19.3 | 6.9 | 6.9 |
| Hypromellose (Methocel K15M Premium) | 4.8 | 5.0 | 1.9 | 1.9 |
| Crospovidone (Type A) | — | — | 2.4 | 2.4 |
| Sodium Bicarbonate | — | — | 3.0 | 3.0 |
| Carbomer Homopolymer Type A, (Carbopol 71G) | 0.0 | 0.0 | 3.2 | 3.2 |
| Colorant | 0.1 | 0.1 | 0.1 | 0.1 |
| Magnesium stearate | 0.3 | 0.3 | 0.2 | 0.2 |

Lithium carbonate has been granulated using aqueous povidone solution in a High-Shear Mixer (GMX). The granules were dried using fluid bed dryer until the LOD was less than or equal to 1% was achieved. The dried granulation was milled through a Fitzmill obtain API granules.

The API granules were blended with extra-granular materials and then lubricated using magnesium stearate using a blender to obtain the API layer blend. This final blend was used in the compression of manufacturing of the bilayer tablet as the API layer.

The placebo layer blend was prepared by blending the placebo layer materials and the blend was lubricated using magnesium stearate in a blender to obtain the placebo layer blend. This final blend was used in the compression of manufacturing of the bilayer tablet as the placebo layer.

The API layer blend and placebo layer blend were compressed using a rotary tablet press to obtain bilayer tablets with target hardness of approximately 20 kp. The bilayer tablet cores could then be film-coated using Opadry to obtain film coated bilayer tablets.

### Example 6. Dissolution Study

The extended-release lithium carbonate matrix tablet (non gastroretentive) of Example 1 was analyzed in a dissolution study utilizing USP Apparatus 1 (Baskets) at 100 rpm in pH 4.5 acetate buffer (900 mL; 37.0° C.±0.5° C.) as dissolution medium. Dissolution samples were collected at 2, 4, 6, 8, 10, 12, 14, 16, 20 and 24 hours and analyzed with HPLC employing conductivity detection by comparing the peak response in the samples to that of a standard solution with known concentration of lithium. The mean dissolution data are provided in the table below, along with the dissolution results for a comparative 450 mg lithium carbonate extended-release tablet product (ANDA 076691 Hikma Pharmaceuticals USA Inc).

| | | Example 1 Lithium |
|---|---|---|
| | Lithium Carbonate | Carbonate Tablet, |
| Time | Tablet, 450 mg | 900 mg |
| (hr) | % released | |
| 2 | 44 | 23 |
| 4 | 68 | 48 |
| 6 | 84 | 70 |
| 8 | 95 | 88 |
| 10 | 101 | 98 |
| 12 | 100 | 102 |
| 14 | 100 | 103 |
| 16 | 99 | 102 |
| 20 | 99 | 102 |
| 24 | 99 | 102 |

### Example 7. Dissolution Study

The extended-release lithium carbonate matrix bilayer tablets (gastro-retentive) of Examples 2-5 were analyzed in a dissolution study utilizing USP Apparatus 3 (Reciprocating Cylinder) at 20 dips per minute (DPM) in pH 4.5 acetate buffer (250 mL; 37.0° C.±0.5° C.) as dissolution medium. Dissolution samples were collected at 0.5, 1, 2, 3, 4, 6, 8, 10 and 12 hours and analyzed with HPLC employing conductivity detection by comparing the peak response in the samples to that of a standard solution with known concentration of lithium. The mean dissolution data are provided in the table below, along with the dissolution results for a comparative 450 mg lithium carbonate extended-release tablet product (ANDA 076691 Hikma Pharmaceuticals USA Inc).

| | Lithium | | | | |
|---|---|---|---|---|---|
| | Carbonate | Lithium Carbonate Tablet, 900 mg | | | |
| Time | Tablet, 450 mg | Example 2 | Example 3 | Example 4 | Example 5 |
| (hr) | % released | | | | |
| 0.5 | 34 | 17 | 19 | 11 | 11 |
| 1 | 60 | 20 | 35 | 24 | 21 |
| 2 | 96 | 45 | 63 | 49 | 42 |
| 3 | 105 | 61 | 81 | 64 | 58 |
| 4 | 107 | 76 | 90 | 75 | 74 |
| 6 | 106 | 88 | 95 | 82 | 80 |
| 8 | 106 | 92 | 101 | 89 | 85 |
| 10 | 106 | 98 | 102 | 94 | 89 |
| 12 | 106 | 100 | 103 | 100 | 93 |

Examples 2-5 are gastroretentive bilayer tablet formulations. It is expected that the placebo layer will swell and promote gastric retention, while the API layer continues to release lithium carbonate by erosion and diffusion. Using the gastroretentive formulation, it is expected that all of the lithium carbonate is released within the absorption window to allow for a complete absorption of lithium into the system of the subject.

### Examples 8-10. Extended-Release Lithium Carbonate Matrix Tablet (Non Gastroretentive) and Matrix Bilayer (Gastroretentive) Tablets

Example 8.

| Granulation | % |
|---|---|
| Lithium carbonate | 69.7 |
| Povidone | 2.9 |
| Microcrystalline cellulose (AVICEL PH | 12.4 |
| 101) | |
| Hypromellose (HPMC K100LV CR) | 5.4 |
| Hypromellose (HPMC K15M Premium) | 5.8 |
| Colloidal silicon dioxide (Cab-O-Sil, | 0.5 |
| M5P) | |
| Magnesium stearate | 0.8 |
| Film coating | 2.4 |
| Purified water | — |

Example 8 lithium carbonate matrix tablets were prepared in a similar process to Example 1; the intragranular ingredients included povidone, lithium carbonate (API), and microcrystalline cellulose. The granules were dried to LOD of less than or equal to 0.5% to form Lithium carbonate Granules. The Lithium carbonate Granules were blended with extragranular ingredients microcrystalline cellulose, the hypromellose ingredients, colloidal silicon dioxide, and magnesium stearate. Single layer tablets were compressed using rotary tablet press as per target tablet weight. The core tablets were then film-coated using an Opadry II film coating.

| | Example 9. | Example 9a. | Example 10. |
|---|---|---|---|
| | % | | |
| API Granules | | | |
| Lithium carbonate | 55.9 | 49.4 | 59.3 |
| Povidone | 2.2 | 2.0 | 2.4 |
| Extended release Blend | | | |
| Microcrystalline cellulose (AVICEL PH 101) | 2.5 | 2.2 | — |
| Polyethylene oxide (WSRN12K LEO) | — | — | 9.9 |
| Hypromellose (HPMC K4M Premium CR) | 8.1 | 7.1 | — |
| Hypromellose (HPMC K100 Premium LV CR) | 4.0 | 3.6 | — |
| Colloidal silicon dioxide | 0.2 | 0.2 | — |
| Magnesium stearate | 0.6 | 0.6 | 0.5 |
| Gastroretentive Layer | | | |
| Polyethylene oxide (WSR 303) | 19.4 | 25.7 | 20.6 |
| Hypromellose (Methocel K15M Premium) | 5.1 | 6.8 | 5.4 |
| Colorant | 0.04 | 0.01 | 0.05 |
| Magnesium stearate | 0.3 | 0.4 | 0.3 |
| Total Placebo Layer | 24.9 | 32.9 | 26.4 |
| Film coating | 1.5 | 2.0 | 1.5 |
| Purified water | — | — | — |

Examples 9.-10. extended-release lithium carbonate matrix bilayer tablet, gastroretentive formulations were prepared by processes similar to Examples 2.-5.

### Example 11. Bioavailability Studies, Fed State

Bioavailability studies in the fed state were conducted for the tablets of Examples 8-10. Open label, balanced, single oral dose, randomized, four-period, four-treatment, four sequence, crossover, relative bioavailability study of test formulations of Examples 8-10 (Lithium Carbonate Extended Release Tablets 900 mg) administered once a day compared to Reference formulation Lithium Carbonate Extended-Release Tablets, 450 mg (Hikma Pharmaceuticals USA Inc.) administered twice a day in healthy adult human subjects under fed conditions. The results of the studies are provided in the following Tables. Example 9 once a day extended-release tablet was found to be bioequivalent to currently available 450 mg Lithium Carbonate Extended-Release Tablets twice a day, thereby providing a high strength lithium salt formulation with a satisfactory extended-release profile for reduced number of administrations per day and fewer dosage units per administration for simpler and more convenient dosing. Key benefits of once-a-day formulation include low peak and trough drug concentration or spikes due to single time dosing compared to twice daily dosing. Since the pill burden will reduce to half, the once a day extended-release tablet would significantly enhance patient compliance and treatment adherence.

For Example 8 (T) vs. Reference (R):

| | Geometric Least Squares | | | | |
|---|---|---|---|---|---|
| | Means and its ratio | | | | |
| | Test | Reference | | Intra- | 90% |
| PK | Product | Product | | subject | Confi- |
| Parameters | (T) | (R) | (T/R) | CV | dence |
| (Units) | (N = 16) | (N = 16) | (%) | (%) | Interval |
| Cₘₐₓ | 5357.987 | 3490.536 | 153.50 | 15.32 | 139.23%- |
| (ng/mL) | | | | | 169.23% |
| AUC₀₋ₜ | 97193.989 | 109741.432 | 88.57 | 13.33 | 81.35%- |
| (hr*ng/mL) | | | | | 96.42% |
| AUC_{0-∞} | 101876.634 | 115779.877 | 87.99 | 12.78 | 81.10%- |
| (hr*ng/mL) | | | | | 95.47% |

For Example 9 (T) vs. Reference (R):

| | Geometric Least Squares | | | | |
|---|---|---|---|---|---|
| | Means and its ratio | | | | |
| | Test | Reference | | Intra- | 90% |
| PK | Product | Product | | subject | Confi- |
| Parameters | (T) | (R) | (T/R) | CV | dence |
| (Units) | (N = 15) | (N = 15) | (%) | (%) | Interval |
| Cₘₐₓ | 3613.434 | 3456.896 | 104.53 | 14.41 | 95.19%- |
| (ng/mL) | | | | | 114.79% |
| AUC₀₋ₜ | 106745.753 | 109638.895 | 97.36 | 5.39 | 93.99%- |
| (hr*ng/mL) | | | | | 100.85% |
| AUC_{0-∞} | 111717.543 | 115580.325 | 96.66 | 4.95 | 93.58%- |
| (hr*ng/mL) | | | | | 99.83% |

For Example 10 (T) vs. Reference (R):

| | Geometric Least Squares | | | | |
|---|---|---|---|---|---|
| | Means and its ratio | | | | |
| | Test | Reference | | Intra- | |
| PK | Product | Product | | subject | 90% |
| Parameters | (T) | (R) | (T/R) | CV | Confidence |
| (Units) | (N = 14) | (N = 14) | (%) | (%) | Interval |
| Cₘₐₓ | 3016.836 | 3576.811 | 84.34 | 18.40 | 74.20%- |
| (ng/mL) | | | | | 95.87% |
| AUC₀₋ₜ | 96821.306 | 110167.633 | 87.89 | 31.38 | 70.88%- |
| (hr*ng/mL) | | | | | 108.98% |
| AUC_{0-∞} | 102109.342 | 116583.882 | 87.58 | 31.23 | 70.70%- |
| (hr*ng/mL) | | | | | 108.50% |

### Example 12. Bioavailability Studies, Fasting State

Bioavailability studies in the fasting state were conducted for the tablets of Examples 9-10. Open label, balanced, single oral dose, randomized, three-period, three-treatment, three sequence, three-way crossover, relative bioavailability study of test formulations of Examples 9-10 (Lithium Carbonate Extended Release Tablets 900 mg) administered once a day compared to Reference formulation Lithium Carbonate Extended-Release Tablets, 450 mg (Hikma Pharmaceuticals USA Inc.) administered twice a day in healthy adult human subjects under fasting conditions. The results of the studies are provided in the following Tables.

For Example 9 (T) vs. Reference (R):

| | Geometric Least Squares | | | | |
|---|---|---|---|---|---|
| | Means and its ratio | | | | |
| | Test | | | Intra- | |
| PK | Product | Reference | | subject | 90% |
| Parameters | (T) | Product | (T/R) | CV | Confidence |
| (Units) | (N = 16) | (N = 16) | (%) | (%) | Interval |
| Cₘₐₓ | 2233.460 | 3662.617 | 60.98 | 16.54 | 55.00%- |
| (ng/mL) | | | | | 67.61% |
| AUC₀₋ₜ | 46854.920 | 107193.177 | 43.71 | 22.80 | 37.95%- |
| (hr*ng/mL) | | | | | 50.35% |
| AUC_{0-∞} | 48838.310 | 111563.679 | 43.78 | 22.32 | 38.11%- |
| (hr*ng/mL) | | | | | 50.28% |

For Example 10 (T) vs. Reference (R):

| | Geometric Least Squares | | | | |
|---|---|---|---|---|---|
| | Means and its ratio | | | | |
| | Test | | | Intra- | |
| PK | Product | Reference | | subject | 90% |
| Parameters | (T) | Product | (T/R) | CV | Confidence |
| (Units) | (N = 15) | (N = 15) | (%) | (%) | Interval |
| Cₘₐₓ | 2281.435 | 3644.095 | 62.61 | 9.54 | 58.80%- |
| (ng/mL) | | | | | 66.66% |
| AUC₀₋ₜ | 49546.344 | 104351.474 | 47.48 | 17.88 | 42.23%- |
| (hr*ng/mL) | | | | | 53.38% |
| AUC_{0-∞} | 51500.718 | 108497.299 | 47.47 | 17.71 | 42.27%- |
| (hr*ng/mL) | | | | | 53.30% |

A bioavailability study in the fasting state was conducted for the single layer tablet of Example 8. Open label, balanced, single oral dose, randomized, four-period, four-treatment, four sequence, crossover, relative bioavailability study of test formulation of Example 8 (Lithium Carbonate Extended Release Tablets 900 mg) administered once a day compared to Reference formulation Lithium Carbonate Extended-Release Tablets, 450 mg (Hikma Pharmaceuticals USA Inc.) administered twice a day in healthy adult human subjects under fasting conditions. The results of the study are provided in the following Table.

For Example 8 (T) vs. Reference (R):

| | Geometric Least Squares | | | | |
|---|---|---|---|---|---|
| | Means and its ratio | | | | |
| | Test | Reference | | Intra- | |
| PK | Product | Product | | subject | 90% |
| Parameters | (T) | (R) | (T/R) | CV | Confidence |
| (Units) | (N = 22) | (N = 22) | (%) | (%) | Interval |
| Cₘₐₓ | 4413.7 | 3977.0 | 110.98 | 11.85 | 104.29%- |
| (ng/mL) | | | | | 118.09% |
| AUC₀₋ₜ | 95539.0 | 120604.0 | 79.22 | 18.70 | 71.86%- |
| (hr*ng/mL) | | | | | 87.33% |
| AUC_{0-∞} | 100534.5 | 127211.7 | 79.03 | 18.77 | 71.66%- |
| (hr*ng/mL) | | | | | 87.16% |

### Example 13. Additional Clinical Studies

A randomized, three-way crossover study evaluating the bioavailability of a single dose once-daily Lithium Carbonate Extended-Release (XR) bi-layer matrix formulation substantially the same as Example 9 compared with two-times-daily and three-times-daily lithium regimens in healthy adult volunteers under fed conditions was conducted.

Purpose: The bioavailability of the once-daily (QD) Lithium Carbonate Extended-Release (XR) 900 mg formulation was compared with two approved reference regimens: twice daily (BID) lithium carbonate ER 450 mg and three times daily (TID) lithium carbonate immediate-release (IR) 300 mg. The primary objective was to compare rate and extent of absorption following a single dose under fed conditions. Safety and tolerability were also assessed.

Methods: This open-label, balanced, randomized, three-period, three-treatment, six-sequence, three-way crossover study was conducted in healthy adult volunteers. Participants received a 900 mg dose of each formulation under fed conditions per assigned treatment sequence. Total study duration was 37 days with a washout period of at least 14 days between each dosing period. Serial blood samples were collected for pharmacokinetic (PK) evaluation. Plasma lithium concentrations were quantified using a validated ICP-OES method. PK parameters included Cₘₐₓ, AUC₀₋ₜ, and AUC_{0-∞}. Bioequivalence was assessed using ln-transformed PK parameters and 90% CIs for geometric least-squares mean ratios.

Results: PK analyses included 25 participants who completed ≥2 study periods. Mean (±SD) Cₘₐₓ values were 4067.9±817.3 ng/mL for QD XR, 4049.8±670.0 ng/mL for the BID ER reference, and 3708.3±540.6 ng/mL for the TID IR reference. Corresponding AUC₀₋ₜ values were 125,690.9±31,738.9, 128,152.5±21,257.3, and 117,254.1±17,645.9 hr*ng/mL, respectively. Median Tₘₐₓ of 12 hour was observed for QD XR formulation compared to 18.0 hour 450 mg ER BID and 20.0 hour 300 mg IR TID after consecutive dosing. For both test-to-reference comparisons, geometric least-squares mean ratios and 90% CIs for Cₘₐₓ, AUC₀₋ₜ, and AUC_{0-∞}, were all within the bioequivalence acceptance criteria of 80.0%-125.0%. Study treatments were well tolerated.

CONCLUSIONS: The QD lithium carbonate XR formulation was bioequivalent to both BID ER and TID IR lithium carbonate under fed conditions. These findings eliminate fluctuations in trough and peak plasma concentrations observed with multiple dosing and support the simplified 900 mg XR QD dosing regimen of lithium carbonate compared with multiple dosing of the approved formulation of 450 mg ER BID and 300 mg IR TID.

### Example 14. Additional Clinical Studies

A randomized, two-way crossover study evaluating the steady-state bioequivalence of a once-daily Lithium Carbonate Extended-Release (XR) 900 mg bi-layer matrix formulation substantially the same as Example 9 compared with two-times-daily lithium carbonate extended-release 450 mg in healthy adults under fed conditions was conducted.

Purpose: This study evaluated the bioequivalence of the once-daily (QD) Lithium Carbonate Extended-Release (XR) 900 mg formulation compared with a twice daily (BID) lithium carbonate extended-release (ER) 450 mg reference product. The primary objective was to demonstrate steady-state bioequivalence of the QD XR test formulation with the approved BID formulation under fed conditions. Safety and tolerability were also assessed.

Methods: This was an open-label, balanced, randomized, two-treatment, two-sequence, two-period, two-way crossover bioequivalence study conducted in healthy adults. Study participants received one treatment option for eight consecutive days under fed conditions, followed by crossover to the alternate treatment. A total of 49 blood samples per subject were collected for pharmacokinetic (PK) evaluation. Plasma lithium concentrations were quantified using a validated ICP-OES method. Primary PK parameters included C_{max,ss}, AUC_{0-τ,ss}, and C_{min,ss}. Bioequivalence was assessed using ln-transformed PK parameters and 90% CIs for geometric least-squares mean ratios.

Results: PK analyses included 31 participants who completed both study periods. Steady state was achieved for all participants for both treatments before the start of full PK assessments on Day 8. Mean (±SD) C_{max,ss} values were 6889.1±1548.9 ng/mL for the QD XR 900 mg formulation and 6747.3±1370.7 ng/mL for the BID ER 450 mg reference formulation. Mean AUC_{0-τ,ss} values were 119,593.6±27,858.4 hr*ng/mL and 122,017.4±25,590.3 hr*ng/mL, respectively. Median T_{max,ss} occurred at 8.0 hour for the XR test formulation and 5.0 hour for the ER reference product. Geometric least-squares mean T/R ratios were 101.45% for C_{max,ss}, 97.30% for AUC_{0-τ,ss}, and 86.84% for C_{min,ss}. The 90% CIs for all ln-transformed primary PK parameters were within the bioequivalence acceptance criteria of 80.0%-125.0%. Study treatments were well tolerated under fed conditions.

CONCLUSIONS: The QD lithium carbonate XR 900 mg formulation was bioequivalent to FDA approved BID lithium carbonate ER 450 mg at steady state under fed conditions following multiple-dose administration (8 days). These findings support the pharmacokinetic comparability of a smoother single daily peak/trough PK profile and simplified QD dosing regimen.

### Example 15. Additional Clinical Studies

A randomized crossover study evaluating dose proportionality of a Lithium Carbonate Extended-Release (XR) 600 mg bi-layer matrix formulation substantially the same as Example 9a and Lithium Carbonate Extended-Release (XR) 900 mg bi-layer matrix formulation substantially the same as Example 9 under fed conditions in healthy adults was conducted.

Purpose: The dose proportionality of the once-daily (QD) Lithium Carbonate Extended-Release (XR) 600 mg and 900 mg formulations was evaluated following a single oral dose under fed conditions in healthy adults. Safety and tolerability were also assessed.

Methods: This was an open-label, balanced, randomized, two-treatment, four-period, four-sequence crossover study conducted in healthy adults. This analysis evaluated dose proportionality for 600 mg and 900 mg XR formulation following single-dose administration under fed conditions only. Blood samples (25 per subject, for 21 subjects) were collected for pharmacokinetic (PK) evaluation. Plasma lithium concentrations were quantified using a validated ICP-OES method. PK parameters included Cₘₐₓ, AUC₀₋ₜ, and AUC_{0-∞}. Dose proportionality was concluded if the 90% CIs for geometric least-squares mean ratios (T2/T1) of dose-normalized Cₘₐₓ, AUC₀₋ₜ, and AUC_{0-∞} were all within the predefined acceptance range of 80.0%-125.0%. Safety was assessed from the screening period to the end of the study, which included clinical examination, clinical laboratory measures, and monitoring for adverse events (AEs).

Results: PK analyses included 21 subjects who completed both treatment periods. Geometric least-squares mean ratios (T2/T1) for dose-normalized PK parameters were 97.65% for Cₘₐₓ (90% CI: 90.48%-105.39%), 105.63% for AUC₀₋ₜ(90% CI: 102.68%-108.67%), and 104.69% for AUC_{0-∞}, (90% CI: 102.39%-107.05%). All 90% CIs for the In-transformed, dose-normalized primary PK parameters were within the acceptance criteria of 80.0%-125.0%, demonstrating proportional increases in systemic exposure between the 600 mg and 900 mg doses. Both dose levels were well tolerated.

CONCLUSIONS: The QD lithium carbonate bi-layer matrix XR formulation demonstrated dose proportionality between 600 mg and 900 mg under fed conditions in healthy adults. These findings support predictable pharmacokinetics across the evaluated doses and provide a pharmacokinetic rationale for flexible dose selection.

In an embodiment, a method of treatment comprises administering an extended-release, bilayer oral tablet once daily with food to a subject in need thereof, wherein the Cₘₐₓ and AUC of lithium upon oral administration of the extended-release, bilayer oral tablet increases dose proportionally from 600 mg to 900 mg. Within this embodiment, the subject is treated for depression or mania.

### Example 16. Population Pharmacokinetic Modeling

OBJECTIVES: A population pharmacokinetic (popPK) model of lithium carbonate was developed to simulate PK profiles of the once-daily Lithium Carbonate Extended Release Tablets (XR) in adult and pediatric patients. Objectives included characterization of lithium pharmacokinetics, evaluation of formulation-switching strategies from two-times-daily (BID) extended-release (ER) and three-times-daily (TID) immediate-release (IR) formulations to once-daily (QD) XR dosing, and assessment of dosing strategies in pediatric and adolescent patients aged 7 to <18 years using fixed allometric scaling.

METHODS: A popPK analysis was conducted using lithium concentration data from three Phase 1 studies in healthy adults evaluating lithium QD XR, BID ER, and TID IR formulations. A two-compartment model was developed to characterize lithium exposure and sources of variability across formulations and dosing regimens. Key covariates, including body weight, food status, and circadian rhythm effects on absorption, were incorporated to support clinically relevant comparisons. The final model simulated steady-state exposure, formulation-switching scenarios, and dosing outcomes in virtual adult and pediatric populations under fed conditions.

RESULTS: The model adequately described lithium concentration-time profiles across different formulations, doses, and food states. Simulations showed dose-proportional increases in steady-state exposure with QD XR dosing. Switching from BID ER or TID IR to QD XR at equivalent total daily doses produced minimal changes in AUC, Cₘₐₓ, and Cₘᵢₙ, with exposures within the expected therapeutic range. Pediatric simulations incorporating allometric scaling predicted higher trough concentrations at higher approved doses, particularly in adolescents at 1800 mg daily, and in children weighing 20 to <30 kg at 1200-1500 mg daily, supporting current weight-based dosing recommendations and maximum dose limits.

CONCLUSION: This popPK model supports QD lithium carbonate XR dosing in adults and pediatric patients and predicts negligible changes in systemic exposure when switching from currently available IR or ER formulations to XR formulation at steady state across approved adult dose ranges.

In an embodiment, switching to the once daily XR formulation (e.g., 600 mg or 900 mg lithium carbonate) from either an immediate release formulation (e.g. 300 mg lithium carbonate per dosage unit) or from a 450 mg lithium carbonate extended release formulation at steady-state across the range of approved doses in adults is predicted to have a negligible impact on lithium Cₘₐₓ, AUC₍₀₋₂₄₎, and Cₘᵢₙ.

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

In general, the disclosure may alternately comprise, consist of, or consist essentially of, any appropriate components herein disclosed. The disclosure may additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

The endpoints of all ranges directed to the same component or property are inclusive of the endpoints, are independently combinable, and include all intermediate points and ranges (e.g., ranges of “up to 25 wt %, or more specifically 5 to 20 wt %” is inclusive of the endpoints and all intermediate values of the ranges of “5 to 25 wt %,” such as “10 to 23 wt %,” “20 to 24,” “1 to 5 wt %,” etc.). Disclosure of a narrower range or more specific group in addition to a broader range is not a disclaimer of the broader range or larger group.

“Combination” is inclusive of blends, mixtures, reaction products, and the like.

The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

Unless otherwise specified herein, any reference to standards, regulations, testing methods and the like, refer to the standard, regulation, guidance or method that is in force at the time of filing of the present application.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of treating a pediatric subject in need thereof, comprising:
administering an extended-release, bilayer oral tablet once daily with food to the pediatric subject to treat depression or mania;
the extended-release, bilayer oral tablet comprises
a first layer comprising an extended-release polymer matrix portion comprising about 600 mg or about 900 mg of lithium carbonate and a controlled-release polymer; and
a second layer comprising a gastroretentive portion comprising a swellable polymer;
wherein the extended-release polymer matrix portion to the gastroretentive portion has a weight ratio of about 4:1 to about 1.5:1.

2. The method of claim 1, wherein the pediatric subject is an adolescent aged 7 to <18 years receiving up to 1800 mg daily maximum dose, or a child weighing 20 to <30 kg receiving up to 1500 mg daily maximum dose.

3. The method of claim 1, wherein the weight ratio of the extended-release polymer matrix portion to the gastroretentive portion is about 3.5:1 to about 2.5:1.

4. The method of claim 1, wherein the lithium carbonate is present in an amount of about 60 to about 90 wt % based on the total weight of the extended-release polymer matrix portion.

5. The method of claim 1, wherein the controlled-release polymer is present in the extended-release polymer matrix portion in an amount of about 8 to about 40 wt % based on the total weight of the extended-release polymer matrix portion.

6. The method of claim 1, wherein the controlled-release polymer is a cellulose ether.

7. The method of claim 1, wherein the gastroretentive portion comprises about 75 to about 99 wt % swellable polymer.

8. The method of claim 1, wherein the swellable polymer is a high molecular weight polyethylene oxide, a cellulose ether, or a combination thereof.

9. The method of claim 1, wherein administering the extended-release, bilayer oral tablet once daily with food to the subject reduces or eliminates fluctuations in trough and peak lithium plasma concentrations compared to 2 or 3 times a day dosing at equivalent total daily doses of lithium carbonate.
